(19) United States
(12) Patent Application Publication
(10) Pub. No.: US 2025/0119703 A1
(43) Pub. Date: Apr. 10, 2025

(54) USER SPECIFIC AUDITORY PROFILES

(71) Applicant: Sound United, LLC, Irvine, CA (US)

(72) Inventors: Brendon Stead, Carlsbad, CA (US); Matthew Patrick Lyons, York, PA (US); Bradley M. Starobin, Baltimore, MD (US); Massi Joe E. Kiani, Laguna Niguel, CA (US)

(21) Appl. No.: 18/903,481

(22) Filed: Oct. 1, 2024

Related U.S. Application Data
(60) Provisional application No. 63/587,857, filed on Oct. 4, 2023.

(51) Int. Cl. H04S 7/00; G06V 40/10; G16H 10/60; H04R 5/02

(52) U.S. Cl. CPC H04S 7/307; G06V 40/10; G16H 10/60; H04R 5/02; H04S 7/303; H04R 2499/13; H04S 2400/11; H04S 2400/13

(57) ABSTRACT

The present disclosure provides systems, methods, and devices for optimizing a user's audio experience. A hardware processor can access one or more transfer functions which can include a user's hearing transfer function and an environmental transfer function. The processor can modify an audio signal based on at least the one or more transfer functions. The processor can determine a user's location. The processor can generate beamforming data based on at least the user's location. The processor can cause one or more speakers to emit audio based at least on the modified audio signal according to the beamforming data to generate a steered array in a direction of the user's location.

### CROSS REFERENCE TO RELATED APPLICATIONS

Any and all applications for which a foreign or domestic priority claim is identified in the Application Data Sheet as filed with the present application are hereby incorporated by reference under 37 CFR 1.57 for all purposes and for all that they contain.

### TECHNICAL FIELD

The present disclosure relates to the field of audio devices and audio signal processing.

### BACKGROUND

Audio devices such as auricular devices emit audio which can be heard by users. Different individuals may have different auditory profiles or transfer functions depending on their unique physiological characteristics such as inner ear characteristics. Different environments may have different transfer function depending on acoustic characteristics of the environments.

### SUMMARY

The present disclosure provides systems, devices, and methods for optimizing a user's audio experience. A user's individual auditory profile which can include and/or be represented as one or more transfer functions can be shared across a plurality of devices associated with the user. The various audio devices can modify an audio signal based at least in part on the user's individual hearing transfer function such that the user's audio experience is optimized for that user for each of the audio devices associated with the user. Audio devices can modify an audio signal based on an environmental transfer function to optimize an audio experience based on the acoustic characteristics of the environment. Audio devices can emit audio using steered arrays (e.g., according to beam patterns). Audio devices can emit audio using a steered array toward a user based on determining the user's location within an environment.

Disclosed herein is an audio system that can comprise one or more hardware processors that can be configured to: access a hearing transfer function originating from an auricular device, wherein the hearing transfer function is generated from audiometry data associated with a user, wherein the hearing transfer function is configured to correct audio playback to account for differences between a hearing perception of the user and normal hearing perception; determine a spatial location of a user within an environment from sensor data; based on the spatial location of the user, generate beamforming data for generating audio according to a beam pattern having an acoustic lobe at the spatial location of the user; modify an audio playback signal based on the hearing transfer function; and cause one or more speakers to emit modified audio based on the modified audio playback signal with the beamforming data to cause the acoustic lobe to form at the spatial location of the user with the modified audio to account for the hearing perception of the user.

In some implementations, the one or more hardware processors are configured to: determine an updated spatial location of the user within the environment from the sensor data as the user moves throughout the environment; and generate the beamforming data for generating the audio having the acoustic lobe at the updated spatial location of the user as the user moves throughout the environment.

In some implementations, the one or more hardware processors are configured to: generate the beamforming data based on the spatial location of the user and another spatial location of another user, the beam pattern having the acoustic lobe at the spatial location of the user and another acoustic lobe at the another spatial location of the another user; and cause the one or more speakers to emit the audio based on the beamforming data to cause the acoustic lobe to form at the spatial location of the user with the modified audio and to cause the another acoustic lobe to form at the another spatial location of the another user without the modified audio.

In some implementations, the audiometry data includes one or more of DPgram data or audiogram data.

In some implementations, the sensor data originates from one or more of a camera, mmWave sensor, or ultra-wide band (UWB) sensor.

In some implementations, the sensor data comprises image data, and the one or more hardware processors are configured to: determine an identity of the user from the image data with one or more image processing techniques; and update the beamforming data as the user moves throughout the environment to update the beam pattern with the acoustic lobe continuously at the spatial location of the user.

In some implementations, the one or more hardware processors are configured to: analyze the sensor data to determine an identity of the user; and access the hearing transfer function from memory based on determining that the identity of the user corresponds to the hearing transfer function.

In some implementations, the one or more hardware processors are configured to: access the hearing transfer function from the auricular device over a wireless communication network based on determining that the auricular device and the audio system are associated with the user.

In some implementations, the one or more speakers are positioned in a vehicle, and the one or more hardware processors are configured to access the hearing transfer function from the auricular device in response to the auricular device being positioned within a receptacle of the vehicle.

In some implementations, the one or more hardware processors are configured to access the hearing transfer function from an electronics medical records (EMR) database over a network.

In some implementations, the one or more hardware processors are configured to access a hearing transfer function associated with each ear of the user.

In some implementations, the one or more hardware processors are configured to: access an environmental transfer function associated with an environment of the user, wherein the environmental transfer function is based on one or more acoustic characteristics of the environment determined from an audio spectral response; and modify the audio playback signal based on the environmental transfer function.

In some implementations, the one or more hardware processors are configured to apply one or more device filters to the audio playback signal to modify the audio playback signal to account for physical characteristics of the audio system that affect an acoustic quality of audio playback from the one or more speakers.

In some implementations, the one or more hardware processors are configured to modify the audio playback signal based on applying one or more frequency dependent gains to an amplitude of the audio playback signal.

In some implementations, the one or more hardware processors are configured to modify the audio playback signal based on adjusting one or more of a phase of the audio playback signal, a latency of the audio playback signal, or an amplitude of the audio playback signal.

Disclosed herein is a computer-implemented method comprising: accessing a hearing transfer function originating from an auricular device, wherein the hearing transfer function is generated from audiometry data associated with a user, wherein the hearing transfer function is configured to correct audio playback to account for differences between a hearing perception of the user and normal hearing perception; determining a spatial location of a user within an environment from sensor data; based on the spatial location of the user, generating beamforming data for generating audio according to a beam pattern having an acoustic lobe at the spatial location of the user; modifying an audio playback signal based on the hearing transfer function; and causing one or more speakers to emit modified audio based on the modified audio playback signal with the beamforming data to cause the acoustic lobe to form at the spatial location of the user with the modified audio to account for the hearing perception of the user.

Disclosed herein is non-transitory computer-readable media including computer-executable instructions that, when executed by a computing system, cause the computing system to perform operations comprising: accessing a hearing transfer function originating from an auricular device, wherein the hearing transfer function is generated from audiometry data associated with a user, wherein the hearing transfer function is configured to correct audio playback to account for differences between a hearing perception of the user and normal hearing perception; determining a spatial location of a user within an environment from sensor data; based on the spatial location of the user, generating beamforming data for generating audio according to a beam pattern having an acoustic lobe at the spatial location of the user; modifying an audio playback signal based on the hearing transfer function; and causing one or more speakers to emit modified audio based on the modified audio playback signal with the beamforming data to cause the acoustic lobe to form at the spatial location of the user with the modified audio to account for the hearing perception of the user.

A non-transitory computer-readable media can include computer-executable instructions that, when executed by a computing system, can cause the computing system to perform operations. The operations can comprise receiving, via a network, auditory profile information from an audio device configured to measure an auditory profile of a user, wherein the auditory profile information is associated with one or more ears of the user. The operations can comprise on at least analyzing user data and device data. The operations can comprise communicating, via the network, the auditory profile information to the one or more auxiliary audio devices to cause the one or more auxiliary audio devices to adjust an audio playback based on at least the auditory profile information.

In some implementations, the auditory profile information comprises one or more hearing transfer functions.

An audio device can generate user location-specific audio. The audio device can comprise one or more hardware computer processors that can be configured to execute a plurality of computer executable instructions to cause the audio device to: access sensor data; analyze the sensor data to determine a spatial location of a user within an environment; generate beamforming data based on at least the spatial location of the user, wherein the beamforming data includes data relating to generating audio at the spatial location of the user according to a beam pattern; and cause one or more speakers to emit audio based at least on an audio playback signal according to the beamforming data.

In some implementations, the sensor data includes image data generated by a camera sensor.

In some implementations, the sensor data includes data generated by a Lidar sensor.

In some implementations, the sensor data includes data generated by a mmWave sensor.

In some implementations, the one or more hardware computer processors are configured to execute the computer executable instructions to cause the audio device to: access additional sensor data; analyze the sensor data to determine an updated spatial location of the user within the environment; generate updated beamforming data based on at least the updated spatial location of the user, wherein the updated beamforming data includes data relating to generating audio at the updated spatial location of the user according to a beam pattern; and cause the one or more speakers to emit audio based at least on the updated beamforming data.

In some implementations, the one or more hardware computer processors are configured to execute the computer executable instructions to cause the audio device to: analyze the sensor data to determine an identity of the user; select the auditory profile information from a plurality of available sets of auditory profile information based at least on the identity of the user; modify an input audio signal based at least on the auditory profile information to produce the audio playback signal.

An audio device can generate user-specific audio. The audio device can comprise: one or more hardware computer processors configured to execute a plurality of computer executable instructions to cause the audio device to: access auditory profile information associated with a user identified in a proximity of the audio device; adjust an audio playback signal based on at least the auditory profile information to produce an adjusted audio playback signal; generate beamforming data based on at least a location of the user within an environment, the beamforming data relating to generating audio according to a beam pattern having one or more lobes in a direction the user; and cause one or more speakers to emit audio based at least on the adjusted audio playback signal according to the beamforming data.

An audio device can generate user-specific audio. The audio device can comprise: one or more hardware computer processors configured to execute a plurality of computer executable instructions to cause the audio device to: access first auditory profile information associated with a first user; adjust an audio playback signal based on at least the first auditory profile information to generate a first adjusted audio playback signal; access second auditory profile information associated with a second user; adjust the audio playback signal based on at least the second auditory profile information to generate a second adjusted audio playback signal; generate beamforming data based on at least a location of the first user and a location of the second user, the beamforming data including data relating to generating a beam pattern having a first acoustic lobe in a direction of the location of the first user and a second acoustic lobe in a direction of the location of the second user; and cause one or more speakers to emit audio based at least on the first adjusted audio playback signal as the first acoustic lobe of the beam pattern according to the beamforming data, and emit audio based at least on the second adjusted audio playback signal as the second acoustic lobe of the beam pattern according to the beamforming data.

An audio device can generate user-specific audio. The audio device can comprise: one or more hardware computer processors configured to execute a plurality of computer executable instructions to cause the audio device to: determine a location of a first user based on at least sensor data generated by a sensor; determine a location of a second user based on at least the sensor data generated by the sensor; generate beamforming data based on at least the location of the first user and the location of the second user, wherein the beamforming data includes data relating to generating sound according to a beam pattern having a first lobe in a direction of the location of the first user and a second lobe in a direction of the location of the second user; and cause one or more speakers to emit sound based at least on an audio playback signal according to the beamforming data.

An audio device can generate user-specific audio. The audio device can comprise: one or more hardware computer processors configured to execute a plurality of computer executable instructions to cause the audio device to: access one or more hearing transfer functions associated with a user; access one or more environmental transfer functions associated with an environment of the user; adjust an audio playback signal based on at least the one or more hearing transfer functions and the one or more environmental transfer functions; and cause one or more speakers to emit audio based at least on the adjusted audio playback signal.

A computing system can comprise: a computer readable storage medium having program instructions embodied therewith; and one or more processors configured to execute the program instructions to cause the computing system to: access auditory profile information associated with a user; access user data associated with the user; access device data; determine one or more devices associated with the user based on at least the user data and the device data; and communicate the auditory profile information to the one or more devices.

In some implementations, the one or more devices comprises one or more computing devices.

In some implementations, the one or more devices comprises one or more audio devices.

In some implementations, the one or more processors is further configured to execute the program instructions to cause the computing system to access the auditory profile information by retrieving the auditory profile information from the computer readable storage medium.

In some implementations, the one or more processors is further configured to execute the program instructions to cause the computing system to determine the auditory profile information based on at least audiometry data.

In some implementations, the one or more processors is further configured to execute the program instructions to cause the computing system to access the auditory profile information by receiving the auditory profile information from an audio device remote to the computing system.

In some implementations, the one or more processors is further configured to execute the program instructions to cause the computing system to access the auditory profile information by receiving the auditory profile information from a server remote to the computing system.

In some implementations, the device data includes one or more of a MAC address or IP address.

In some implementations, the user data includes account information associated with the user.

In some implementations, the auditory profile information includes a hearing transfer function associated with each ear of the user.

In some implementations, the auditory profile information comprises auditory profile information determined most recently.

A computing system can comprise: a computer readable storage medium having program instructions embodied therewith; and one or more processors configured to execute the program instructions to cause the computing system to: access an audio playback signal; access sensor data; determine a user location based on at least the sensor data; generate beamforming data based on at least the user location, wherein the beamforming data includes data relating to generating audio at the user location according to a beam pattern; and cause one or more speakers to emit audio based at least on the audio playback signal according to the beamforming data.

In some implementations, the one or more processors are further configured to execute the program instructions to cause the computing system to continuously determine the user location.

In some implementations, the sensor data includes image data collected by a camera.

In some implementations, the one or more processors are further configured to execute the program instructions to cause the computing system to perform one or more image processing techniques on the image data to determine the user location.

In some implementations, the sensor data includes data collected by a Lidar system.

In some implementations, the sensor data includes data collected by a mmWave system.

A computing system can comprise: a computer readable storage medium having program instructions embodied therewith; and one or more processors configured to execute the program instructions to cause the computing system to: access an audio playback signal; access one or more transfer functions; adjust the audio playback signal based on at least the one or more transfer functions; determine a user location; generate beamforming data based on at least the user location, wherein the beamforming data includes data relating to generating audio signal at the user location according to a beam pattern; and cause one or more speakers to emit audio based at least on the adjusted audio playback signal according to the beamforming data.

In some implementations, the one or more processors are further configured to execute the program instructions to cause the computing system to: access sensor data; and determine the user location based on at least the sensor data.

In some implementations, the one or more transfer functions includes a hearing transfer function associated with a user.

In some implementations, the one or more transfer functions includes an environmental transfer function associated with an environment.

A computing system can comprise a computer readable storage medium having program instructions embodied therewith; and one or more processors configured to execute the program instructions to cause the computing system to: access an audio playback signal; access first auditory profile information associated with a first user; adjust the audio playback signal based on at least the first auditory profile information to generate a first adjusted audio playback signal; determine a location of the first user; access second auditory profile information associated with a second user; adjust the audio playback signal based on at least the second auditory profile information to generate a second adjusted audio playback signal; determine a location of the second user; generate beamforming data based on at least the location of the first user and the location of the second user, wherein the beamforming data includes data relating to generating audio according to a beam pattern having a first lobe in a direction of the location of the first user and a second lobe in a direction of the location of the second user; and cause one or more speakers to emit sound based at least on the first adjusted audio playback signal and the second adjusted audio playback signal according to the beamforming data.

In some implementations, the program instructions are configured to cause the computing system to: analyze sensor data to determine an identify of the first user; selecting the first auditory profile information based at least on the determined identify of the first user; analyze the sensor data to determine an identify of the second user; selecting the second auditory profile information based at least on the determined identify of the second user.

A computing system can comprise: a computer readable storage medium having program instructions embodied therewith; and one or more processors configured to execute the program instructions to cause the computing system to: access an audio playback signal; access sensor data; determine a location of a first user based on at least the sensor data; determine a location of a second user based on at least the sensor data; generate beamforming data based on at least the location of the first user and the location of the second user, wherein the beamforming data includes data relating to generating audio according to a beam pattern having a first lobe in a direction of the location of the first user and a second lobe in a direction of the location of the second user; and cause one or more speakers to emit audio based at least on the audio playback signal according to the beamforming data.

A computing system can comprise: a computer readable storage medium having program instructions embodied therewith; and one or more processors configured to execute the program instructions to cause the computing system to: access an audio playback signal; access one or more hearing transfer functions associated with a user; access one or more environmental transfer functions associated with an environment; adjust the audio playback signal based on at least the one or more hearing transfer functions and the one or more environmental transfer functions; and cause one or more speakers to emit audio based at least one the adjusted audio playback signal.

In some implementations, the one or more processors are further configured to execute the program instructions to cause the computing system to: determine a user location; generate beamforming data based on at least the user location, wherein the beamforming data includes data relating to generating audio using a steered array according to a beam pattern; and cause the one or more speakers to emit audio based at least on the adjusted audio playback signal according to the beamforming data.

In some implementations, the one or more processors are further configured to execute the program instructions to cause the computing system to: access sensor data; and determine the user location based on at least the sensor data.

In some implementations, the one or more processors are further configured to execute the program instructions to cause the computing system to determine the one or more environmental transfer functions based on at least one or more acoustic characteristics of the environment.

In some implementations, the one or more processors are further configured to execute the program instructions to cause the computing system to determine the one or more environmental transfer functions based on at least a spectral response of the environment.

In some implementations, the one or more processors are further configured to execute the program instructions to cause the computing system to determine the one or more environmental transfer functions based on at least performing an “in-room equalization” of the environment.

In some implementations, the one or more processors are further configured to execute the program instructions to cause the computing system to adjust the audio playback signal by applying one or more filters to the audio playback signal.

In some implementations, the one or more processors are further configured to execute the program instructions to cause the computing system to adjust the audio playback signal by adjusting a phase of the audio playback signal.

In some implementations, the one or more processors are further configured to execute the program instructions to cause the computing system to adjust the audio playback signal by adjusting a latency of the audio playback signal.

In some implementations, the one or more processors are further configured to execute the program instructions to cause the computing system to adjust the audio playback signal by adjusting an amplitude of the audio playback signal.

In some implementations, the one or more processors are further configured to execute the program instructions to cause the computing system to adjust the audio playback signal by applying one or more frequency dependent gains to an amplitude of the audio playback signal.

An audio device can comprise: one or more hardware processors configured to: generate auditory profile information for a user based at least on an otoacoustic emission from the user's ear; determine a proximity of an additional audio device to the audio device; and in response to at least determining the proximity of the additional audio device to the audio device, communicate the auditory profile information to the additional audio device.

In some implementations, the one or more hardware processors are further configured to communicate the auditory profile information to the additional audio device in response to at least a user input.

In some implementations, the one or more hardware processors are further configured to communicate the auditory profile information to the additional audio device in response to at least verifying an identity of the user device.

In some implementations, the one or more hardware processors are further configured to communicate the auditory profile information to the additional audio device via one or more wireless communication protocols.

In some implementations, the audio device further comprises one or more speakers configured to emit an audio playback based on at least the auditory profile information.

In some implementations, the audio device further comprises one or more speakers configured to emit sound configured to induce the otoacoustic emission from the user's ear; and a sensor configured to measure the otoacoustic emission from the user's ear.

In some implementations, the audio device comprises ear buds.

In some implementations, the additional audio device is a vehicle sound system of a vehicle.

In some implementations, the vehicle includes a receiver configured to receive the audio device, and wherein the audio device is configured to communicate the auditory profile information to the vehicle sound system in response to the audio device being positioned in the receiver.

In some implementations, the receiver comprises a cradle.

In some implementations, the vehicle comprises a port, and wherein the audio device is configured to communicate the auditory profile information to the vehicle sound system in response to the audio device being coupled to the port of the vehicle.

In some implementations, the auditory profile information includes at least one hearing transfer function.

A method for transferring auditory profile information between audio devices can comprise: accessing auditory profile information stored on a first audio device associated with a user; identifying a second audio device associated with the user; establishing a communication between the first audio device and the second audio device; and communicating the auditory profile information from the first audio device to the second audio device via the communication.

In some implementations, the auditory profile information comprises one or more of physiological data, audiometry data, or one or more hearing transfer functions.

In some implementations, establishing the communication between the first audio device and the second audio device comprises establishing a wireless communication protocol between the first audio device and the second audio device.

In some implementations, establishing the communication between the first audio device and the second audio device comprises establishing a wired connection between the first audio device and the second audio device.

In some implementations, the first audio device comprises one or more auricular devices.

In some implementations, the first audio device comprises a case associated with one or more auricular devices.

In some implementations, the first audio device comprises a mobile audio device comprising a phone.

In some implementations, the first audio device comprises an auricular device, wherein the second audio device comprises an acoustic system associated with a vehicle, wherein establishing the communication between the first audio device and the second audio device comprises establishing a wired connection from the auricular device to the acoustic system associated with the vehicle via an auricular device receptacle of the vehicle.

In some implementations, the first audio device comprises an auricular device, wherein the second audio device comprises an acoustic system associated with a vehicle having a receptacle that is configured to receive the auricular device, wherein communicating the auditory profile information from the first audio device to the second audio device is in response to the auricular device being placed in the receptacle of the vehicle.

In some implementations, the first audio device comprises an auricular device, wherein the second audio device comprises an acoustic system associated with a vehicle having a port, wherein communicating the auditory profile information from the first audio device to the second audio device is in response to the auricular device being coupled to the port of the vehicle.

In some implementations, the method further comprises communicating the auditory profile information from the first audio device to the second audio device in response to verifying the second audio device corresponds to the first audio device.

In some implementations, the method further comprises communicating the auditory profile information from the first audio device to the second audio device in response to verifying an identify of a user associated with the first audio device or the second audio device.

In some implementations, the method further comprises: generating a modified audio playback signal based on the auditory profile information; and emitting audio based at least one the modified audio playback signal from one or more speakers of the first audio device.

In some implementations, the method further comprises: generating a modified audio playback signal based on the auditory profile information; and emitting audio based at least one the modified audio playback signal from one or more speakers of the second audio device.

In some implementations, the method further comprises: measuring an otoacoustic emission from the user's ear; determining the auditory profile information from the measured otoacoustic emission.

Various combinations of the above and below recited features, implementations, and aspects are also disclosed and contemplated by the present disclosure.

Additional implementations of the disclosure are described below in reference to the appended claims, which may serve as an additional summary of the disclosure.

In various implementations, systems and/or computer systems are disclosed that comprise a computer-readable storage medium having program instructions embodied therewith, and one or more processors configured to execute the program instructions to cause the systems and/or computer systems to perform operations comprising one or more aspects of the above- and/or below-described implementations (including one or more aspects of the appended claims).

In various implementations, computer-implemented methods are disclosed in which, by one or more processors executing program instructions, one or more aspects of the above- and/or below-described implementations (including one or more aspects of the appended claims) are implemented and/or performed.

In various implementations, computer program products comprising a computer-readable storage medium are disclosed, wherein the computer-readable storage medium has program instructions embodied therewith, the program instructions executable by one or more processors to cause the one or more processors to perform operations comprising one or more aspects of the above- and/or below-described implementations (including one or more aspects of the appended claims).

### BRIEF DESCRIPTION OF THE DRAWINGS

Various implementations will be described hereinafter with reference to the accompanying drawings. These implementations are illustrated and described by example only, and are not intended to limit the scope of the disclosure. In the drawings, similar elements may have similar reference numerals.

FIG. 1 illustrates an example audio device.

FIG. 2 is a block diagram illustrating an example implementation of an audio device.

FIG. 3 is a flowchart illustrating an example process of modifying an audio signal based on a user hearing transfer function.

FIG. 4 a schematic diagram illustrating an example system including one or more audio devices.

FIG. 5 is a flowchart illustrating an example process of communicating hearing transfer function between audio devices.

FIG. 6 is a block diagram illustrating an example audio device.

FIG. 7 illustrates an example audio device.

FIG. 8 is a block diagram illustrating an example implementation of speakers.

FIG. 9 is a schematic diagram illustrating an audio device emitting an example beam pattern having acoustic lobes.

FIG. 10 illustrates an example implementation of an acoustic system including one or more audio devices.

FIG. 11 is a flowchart illustrating an example process for optimizing audio.

### DETAILED DESCRIPTION

The present disclosure will now be described with reference to the accompanying figures, wherein like numerals may refer to like elements throughout. The following description is merely illustrative in nature and is in no way intended to limit the disclosure, its application, or uses. It should be understood that steps within a method may be executed in different order without altering the principles of the present disclosure. Furthermore, the devices, systems, and/or methods disclosed herein can include several novel features, no single one of which is solely responsible for its desirable attributes or which is essential to practicing the devices, systems, and/or methods disclosed herein. Additionally, the structures, systems, and/or devices described herein may be embodied as integrated components or as separate components.

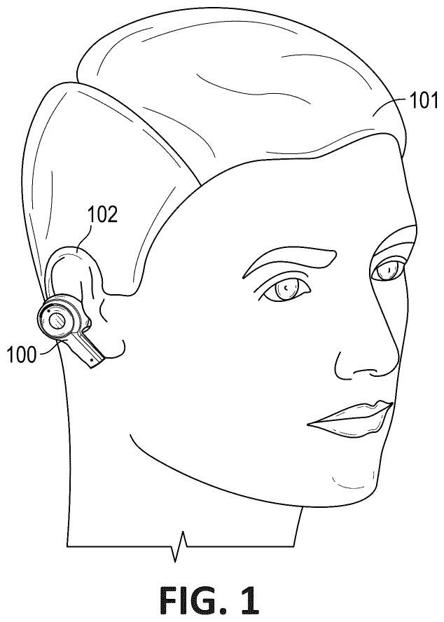

FIG. 1 illustrates an example auricular device 100. The auricular device 100 can be secured to an ear 102 of a user 101 (which may also be referred to as a “subject”, “wearer,” or “patient”). The auricular device 100 can be secured to any of a number of portions and/or locations relative to the ear 102. For example, the auricular device 100 can be secured to, placed adjacent, and/or positioned to be in contact with a pinna, a concha, an ear canal, a tragus, an antitragus, a helix, an antihelix, and/or another portion of the ear 102. In some implementations, the auricular device 100 may be an over-the-ear earcup or headphone. In some implementations, the auricular device 100 may be a hearing aid. In some implementations, the auricular device 100 may be an earbud. In some implementations, a least a portion of the auricular device 100 can be inserted into the external ear canal. In some implementations, two auricular devices 100 can be secured to two ears of the user 101 (e.g., one per each ear). The user 101 may be a newborn baby. The user 101 may be an adult. The auricular device 100 may be used during a diagnostic assessment of acoustic function of the user 101, in some cases. The auricular device 100 may be used for listening to audio (e.g., music, podcast, and the like).

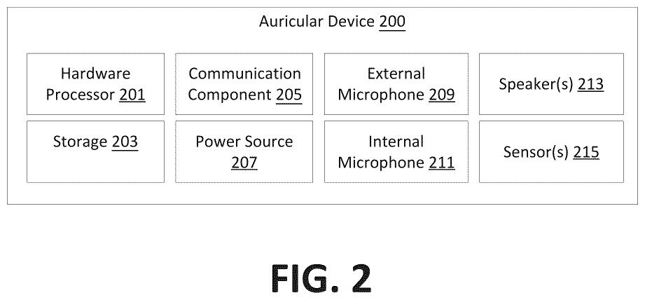

FIG. 2 is a block diagram illustrating an example implementation of an auricular device 200. The auricular device 200 can include similar structural and/or operational features as auricular device 100 shown and/or described herein. The auricular device 200 can include one or more hardware processors 201, a storage 203, a communication component 205, a power source 207, an external microphone 209, an internal microphone 211, one or more speakers 213, and one or more sensors 215. In some implementations, the auricular device 200 may include less than all of the components shown in FIG. 2. For example, the auricular device 200 may not include an external microphone 209, an internal microphone 211, and/or a speaker 213, in some cases. In some implementations, the auricular device 200 may include multiple components of a single component shown. For example, the auricular device 200 may include a plurality of internal and/or external microphones.

One or more components of the auricular device 200 may be integrated as a single unit, such as within a common housing. The auricular device 200 may be one or more of headphones, earphones, earbuds, hearing aids, speakers, sound systems, sound bars, stereo systems, mobile audio devices, portable audio devices, stationary audio devices, mounted audio devices, vehicular audio devices, and the like.

The hardware processor 201 can comprise one or more integrated circuits. The hardware processor 201 can comprise and/or have access to memory. The hardware processor 201 can comprise and/or be embodied as one or more chips, controllers such as microcontrollers (MCUs), and/or microprocessors (MPUs). The hardware processor 201 can comprise a central processing unit (CPU). In some implementations, the hardware processor 201 can be embodied as a system-on-a-chip (SoC). The hardware processor 201 can be configured to implement an operating system which can allow multiple processes to execute simultaneously. The hardware processor 201 can be configured to execute program instructions to cause the auricular device 200 to perform one or more operations. The hardware processor 201 can be configured, among other things, to process data, execute instructions to perform one or more functions, and/or control the operation of the auricular device 200 or components thereof. For example, the hardware processor 201 can process data and can execute instructions to perform functions related to storing and/or transmitting data. In some implementations, the hardware processor 201 may be remote to the auricular device 200. The hardware processor 201 may receive and process data that was collected by the external microphone 209, the internal microphone 211, and/or the sensor(s) 215. The hardware processor 201 can receive and process data from the external microphone 209, the internal microphone 211, and/or the sensor(s) 215. The hardware processor 201 can access data as it is generated in real-time and/or can access data stored in storage 203, such as historical data previously generated.

The hardware processor 201 can execute one or more processes to monitor an auditory health of a user. The hardware processor 201 can generate audiometry data of a user, such as by playing an input audio signal comprising varying amplitudes at a single frequency. The input audio signal can include a test audio signal, and/or a content audio signal comprising music, speech, environment sounds, animal sounds, etc. For example, the input audio signal can include the content audio signal with an embedded test audio signal. The hardware processor 201 can continue to refine audiometry data and/or a hearing transfer function associated with a user, as the user continues to listen to audio.

The hardware processor 201 can implement one or more audiometry tests to assess the operation of an inner ear of a user which may indicate an auditory health of the user. The hardware processor 201 can implement an otoacoustic emissions (OAE) test, such as a stimulus frequency OAE test, swept-tone OAE test, transient evoked OAE test, distortion product OAE test, or pulsed OAE test. The hardware processor 201 can access audio data originating from microphones (e.g., internal microphone 311) to measure otoacoustic emissions (OAE) from the user's ear (e.g., in the external ear canal). The hardware processor 201 can process the OAE audio data from the microphones (e.g., internal microphone 311 during an audiometric test) to generate audiometry data (e.g., processed OAE audio data). Audiometry data can refer to DPgram information, audiogram information, etc., which may also be referred to as auditory profile.

The hardware processor 201 can determine an auditory profile and/or a hearing transfer function associated the user's ear(s) based on audiometry data. The hardware processor 201 can determine a hearing transfer function based on one or more of amplitude, latency, hearing threshold, and/or phase of the measured OAEs. For example, the hardware processor 201 can compare the measured OAEs with response ranges from normal-hearing and hearing-impaired listeners to develop the frequency dependent hearing transfer function for each ear of the user. A hearing transfer function can correlate an actual amplitude or intensity of sound produced by an audio signal to a user-perceived amplitude or intensity. A hearing transfer function can correlate actual amplitudes or intensities to user-perceived amplitudes or intensities for given frequencies of an audio signal. As an example, a hearing transfer function can indicate that an input audio signal that produces a sound at 1000 Hz at 70 dB, is perceived by the user as being at 25 dB. A hearing transfer function can include data to facilitate achieving a certain audio output for a given audio input. For example, the hearing transfer function can include data relating to filters, gain, suppression, phase shift, latency, etc. to apply to an audio signal.

The storage 203 can include any computer readable storage medium and/or device (or collection of data storage mediums and/or devices), including, but not limited to, one or more memory devices that store data, including without limitation, dynamic and/or static random-access memory (RAM), programmable read-only memory (PROM), erasable programmable read-only memory (EPROM), electrically erasable programmable read-only memory (EEPROM), optical disks (e.g., CD-ROM, DVD-ROM, etc.), magnetic disks (e.g., hard disks, floppy disks, etc.), memory circuits (e.g., solid state drives, random-access memory (RAM), etc.), and/or the like. The storage 203 can store data including physiological data, user data, audio data originating from microphones, audiometry data, auditory profiles, and/or hearing transfer functions, for example. The storage 203 can store program instructions that when executed by the hardware processor 201 cause the auricular device 200 to perform one or more operations. Physiological data and/or user data can include user identification data, device identification data, user preferences, medical data, medical records, audiometry data, hearing transfer functions, audio playback data, or the like.

The communication component 205 can facilitate communication (via wireless, wired, and/or wire-like connection) between the auricular device 200 (and/or components thereof) and separate devices, such as separate monitoring hubs, monitoring devices, sensors, systems, servers, or the like. For example, the communication component 205 can be configured to allow the auricular device 200 to wirelessly communicate with other devices, systems, and/or networks over any of a variety of communication protocols, including near-field communication protocols and far-field communication protocols. Near-field communication protocols, which may also be referred to as non-radiative communication, can implement inductive coupling between coils of wire to transfer energy via magnetic fields (e.g., NFMI). Near-field communication protocols can implement capacitive coupling between conductive electrodes to transfer energy via electric fields. Far-field communication protocols, which may also be referred to as radiative communication, can transfer energy via electromagnetic radiation (e.g., radio waves). The communication component 205 can communicate via any variety of communication protocols such as Wi-Fi, Bluetooth®, ZigBee®, Z-wave®, cellular telephony, such as long-term evolution (LTE) and/or 1G, 2G, 3G, 4G, 5G, etc., infrared, radio frequency identification (RFID), satellite transmission, inductive coupling, capacitive coupling, proprietary protocols, combinations of the same, and the like. In some implementations, communication component 205 can implement human body communication (HBC) which can include capacitively coupling a transmitter and receiver via an electric field propagating through the human body. The communication component 205 can allow data and/or instructions to be transmitted and/or received to and/or from the auricular device 200 and separate computing devices. The communication component 205 can be configured to transmit and/or receive (for example, wirelessly) processed and/or unprocessed physiological data with separate computing devices including physiological sensors, other monitoring hubs, remote servers, or the like. As another example, the communication component 205 can be configured to transmit and/or receive (for example, wirelessly) physiological data, audiometry data, and/or playback audio data, with separate computing devices including physiological sensors, other auricular devices, remote servers, or the like. In some implementations, communication component 205 can transfer power required for operation of a computing device. The communication component 205 can be embodied in one or more components that are in communication with each other. The communication component 205 can include one or more of: transceivers, antennas, transponders, radios, emitters, detectors, coils of wire (e.g., for inductive coupling), and/or electrodes (e.g., for capacitive coupling). The communication component 205 can include one or more integrated circuits, chips, controllers, processors, or the like, such as a Wi-Fi chip and/or a Bluetooth chip.

The power source 207 can provide power for components of the auricular device 200. The power source 207 can include a battery (e.g., an internal battery). In some implementations, the power source 207 may be external to the auricular device 200. For example, the auricular device 200 can include or can be configured to connect to a cable which can itself connect to an external power source to provide power to the auricular device 200.

The external microphone 209 may be embodied as part of the auricular device 200. The external microphone 209 may be located within the auricular device 200. The external microphone 209 may be oriented to capture audio originating external to an ear of a user, such as when the external microphone 209 (or the auricular device 200) is donned by the user. The external microphone 209 may be oriented away from a user such as away from an ear of a user. The hardware processor 201 may receive audio data generated by the external microphone 209. The hardware processor 201 can use the audio data to perform noise suppression, such as active noise suppression or adaptive noise suppression.

The internal microphone 211 may be embodied as part of the auricular device 200. The internal microphone 211 may collect physiological data originating from an ear of a user, such as originating from an inner ear and travelling along an ear canal of the user. The internal microphone 211 may be oriented to capture audio originating within an ear of a user, such as within an ear canal, such as when the internal microphone 211 (or the auricular device 200) is donned by the user. The internal microphone 211 may be oriented toward an ear canal of a user. Audio captured by the internal microphone 311 can include audio resulting from activity of hair cells, such as outer hair cells in the cochlea of the user. The audio can include audio resulting from movement of the tectorial membrane of the user. The internal microphone 211 may detect evoked responses such as an acoustic response signal evoked in response to an acoustic stimulus signal. The internal microphone 211 may detect an otoacoustic emission (OAE) originating from within an inner ear of a user. The internal microphone 211 may detect a distortion product otoacoustic emission (DP-OAE), a spontaneous otoacoustic emission (S-OAE), and/or a transient evoked otoacoustic emission (TE-OAE). The hardware processor 201 may receive audio data collected by the internal microphone 211. The hardware processor 201 may use the audio data to determine one or more physiological characteristics of the user. The hardware processor 201 may use the audio data to determine one or more inner ear characteristics of the user. The hardware processor 201 may use the audio data to determine a hearing transfer function of the user.

The speakers 213 may be embodied as part of the auricular device 200. The speakers 213 may emit audio into an ear of a user. The speakers 213 can emit an acoustic stimulus signal, such as to evoke an acoustic response from the inner ear such as an OAE. The speakers 213 can emit sound based on an audio playback signal, such as music or media. The speakers 213 can emit a noise cancelling signal. The hardware processor 201 may generate instructions to control an operation of the speakers 213. The hardware processors 201 may transmit audio data to the speakers 213 to be emitted from the speakers as audio. The speakers 213 can be configured to emit a range of audio frequencies. The speakers 213 can emit multiple frequencies or tones simultaneously. The speakers 213 can include a tweeter, and/or a woofer.

The sensors 215 may be embodied as part of the auricular device 200. In some implementations, the auricular device 200 may include one or more sensors 215 external to the auricular device 200 or remote to the auricular device 200. In some implementations, one or more sensors 215 may be disposed within the auricular device 200 and one or more sensors 215 may be separate from the auricular device 200.

The sensors 215 can include a physiological sensor. The sensors may collect physiological data from a user. The sensors 215 can include one or more of a motion sensor, vibration sensor, accelerometer, gyroscope, force sensor, acoustic sensor, and/or a bone conduction microphone. The sensors 215 may include a transducer configured to convert a signal in one form of energy into another form of energy. The sensors 215 can convert kinetic energy, such as mechanical vibrations, into an electrical signal which may represent the magnitude of the kinetic energy. In some implementations, the sensors 215 may convert kinetic energy originating from within a subject and/or being conducted through the subject into an electrical signal.

The sensors 215 can generate inertial data comprising an electrical signal responsive to detecting internal audio conducted through and/or originating from within the user's body. Internal audio can include kinetic energy such as mechanical vibrations and/or a pressure wave conducted through the user's body tissues. Internal audio can originate from activity of a vocal cord of the subject, activity of respiratory airways of the subject, air movement through respiratory airways of the subject, speaking, breathing, coughing, chewing, swallowing, head movement of the subject, jaw movement of the subject, and/or the like. Inertial data generated by the sensors 215 can indicate one or more frequencies of internal audio including low frequencies, such as frequencies of less than 250 Hz, less than 500 Hz, less than 750 Hz, less than 1,000 Hz, less than 1,250 Hz, less than 1,500 Hz, less than 1,750 Hz, less than 2,000 Hz, etc. Processor 201 can determine frequencies of internal audio from the inertial data generated by sensors 215.

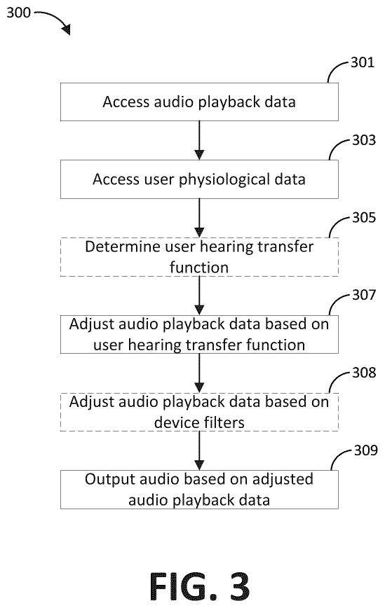

FIG. 3 is a flowchart illustrating an example process 300 of modifying an audio signal based on a user hearing transfer function. This process, in full or parts, can be executed by one or more hardware processors, whether they are associated with a singular or multiple computing devices, and even devices in remote or wireless communication. By way of example, the one or more hardware processors executing process 300 can be associated with auricular device 100/200, a wearable device such as a watch, a mobile device such as a phone, a laptop, a tablet, and/or any of the electronic computing devices shown and/or described herein. The implementations of this process may vary and can involve modifications like omitting blocks, adding blocks, and/or rearranging the order of execution of the blocks. Process 300 serves as an example and is not intended to restrict the present disclosure.

At block 301, a computing device (e.g., one or more hardware processors of a computing device executing program instructions) can access audio playback data (which may also be referred to as audio playback signal). The audio playback data can correspond to a sound desired to be reproduced, such as environment sounds detected at a microphone and/or music or media. The computing device can access the audio playback data from memory. The computing device can receive audio playback data wirelessly via a network. The computing device can generate audio playback data based on data received from a sensor or microphone.

At block 303 the computing device can access physiological data. The physiological data can include audiometry data. The audiometry data can include data relating to and/or originating from one or more of an audiometry test, OAE test, acoustic reflectance test, tympanometry test, eustachian tube test, middle ear reflex test, auditory evoked potentials (AEP), auditory brain stem response (ABR) test, electroencephalography (EEG) test, auditory steady state response (ASSR), etc. The physiological data can include one or more hearing transfer functions. The physiological data can be associated with a user. The physiological data can be associated with an ear of a user. The computing device can access physiological data for each ear of the user. The computing device can access physiological data from memory. The computing device can receive physiological data wirelessly via a network. The computing device can receive physiological data from a remote database, server, computing device, audio device, or the like.

At block 305 the computing device can optionally determine a user hearing transfer function. The computing device can determine the hearing transfer function based on physiological data accessed at block 303, such as audiometry data. In some implementations, the computing device may not need to determine the hearing transfer if it has already been determined and is accessible (e.g., from storage, over the network, etc.).

At block 307 the computing device can adjust the audio playback data or signal based on the transfer function. The computing device can adjust the audio playback data to modify one or more of amplitude, phase, latency of the audio playback data, and/or frequency of audio reproduced by the audio playback data. For example, the computing device can adjust the audio playback data to make frequency-specific adjustments to amplitude, phase, etc. of the audio that is reproduced. The computing device can apply one or more frequency dependent gains to the amplitude of the audio playback data. The computing device can apply one or more filters to the audio playback data. The computing device can adjust the audio playback data by inserting additional audio playback data which may cause constructive and/or destructive interference with audio that is reproduced such as to cause noise suppression. The computing device can adjust the audio playback data so that the user perceives reproduced audio, as if the user had ideal hearing and/or desired hearing.

In one example, the hearing transfer function associated with a user indicates that audio comprising 1000 Hz at 70 dB, is perceived by the user as being at 25 dB. The computing device can determine, based on the transfer function, how to modify audio playback data so that 1000 Hz at 70 dB is perceived by the user as being at 70 dB (e.g., such as by applying frequency specific gains to increase the amplitude or intensity).

In another example, the hearing transfer function associated with a user indicates that audio comprising 1000 Hz at 70 dB, is perceived by the user as being at 60 dB while audio comprising 1500 Hz at 70 dB, is perceived by the user is being at 50 dB. The computing device can determine that in order for the user to perceive the 1000 Hz and 1500 Hz sounds at equal loudness there must be a relative increase in the 1500 Hz sound loudness of 10 dB. The computing device can modify audio playback data accordingly (e.g., so that both 1000 Hz and 1500 Hz sounds are perceived as being at 60 dB). In some cases, the computing device can adjust the relative strength of different frequencies to compensate for a user's specific auditory profile, without adjusting the whole profile to be at the target intensity. Thus, the system can correct for hearing imbalance (e.g., between different frequencies) without fully compensating for general hearing loss or attenuation. The system can flatten, or otherwise adjust, an auditory profile curve, without raising the entire curve to a normal listening level. In some implementations, the system can raise the curve to produce the impression of attenuated hearing.

At block 308 the computing device can optionally adjust the audio playback data based on one or more device filters. Various audio devices may comprise various types or numbers of speakers and may have various physical characteristics that can affect the audio they emit. For example, an over-the-ear headphone, a soundbar, and an in-ear earbud may each emit audio from the same audio playback data. However, the audio that is emitted from these respective devices may differ because these respective devices may each have different speakers and/or physical characteristics that could affect the acoustic characteristics of audio that is reproduced from the same audio playback data. Accordingly, various devices may each have device-specific filters that can be applied to audio playback data to adjust the audio playback data for that specific device, such that audio that is emitted from respective devices has the desired acoustic characteristics, accounting for each device's unique hardware components and characteristics. In some cases, it can be advantageous to apply the hearing correction for the same user differently on different device, or on different types of devices. For example, a hearing correction applied to a set of headphones can be different from a hearing correction applied to a car audio system, which can be different from a hearing correction applied to a home theater audio system, even though each can be for the same user and/or can be based on the same hearing profile or OAE test. In some implementations, the same adjustment based on the user hearing transfer function can be applied at block 307 for different audio devices, but the different hearing devices can apply different device filters at block 308 to adjust the hearing correction to the specific audio device or type of device. In some implementations, the user hearing transfer functions can be adjusted for the different audio devices or types of devices, so that the adjustment at block 307 can be different for different audio devices or types of devices. In some implementations, a frequency response test can be performed on an audio device, and the one or more device filters of block 308 can be based at least in part on the results of the frequency response test. The one or more filters of block 308 can be based on other audio tests or other parameters of the audio device.

At block 309 the computing device can output audio based on the adjusted audio playback data. The computing device can output the sound based on the adjusted audio playback data via one or more speakers. In some implementations, the computing device may output the sound based on the adjusted audio playback data via speakers 213 shown and/or described herein. The computing device can transmit the adjusted audio playback data wirelessly via a network, in some cases, such as to another audio device, which can produce sound based on the adjusted audio playback data.

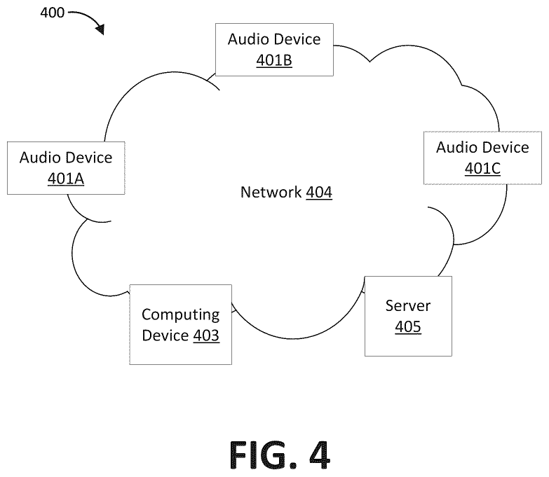

FIG. 4 is a schematic diagram illustrating an example system 400. The system 400 can include one or more computing devices in communication over a network 404, such as audio devices 401 (e.g., 401A, 401B, 401C), computing device 403, and/or server 405. The network 404 can include one or more communications networks. The network 404 can include a plurality of computing devices configured to communicate with one another. The network 404 can include the Internet. The network 404 can include a cellular network. The network 404 can include any combination of a body area network (e.g., implementing human body communication with capacitive coupling via the tissue of a user's body), a local area network (“LAN”) and/or a wide area network (“WAN”), or the like. Accordingly, various computing devices can communicate with one another directly or indirectly via any appropriate communications links and/or networks, such as network 404 (e.g., one or more communications links, one or more computer networks, one or more wired or wireless connections, the Internet, any combination of the foregoing, and/or the like).

Communication over the network 404 can include a variety of communication protocols, including wired communication, wireless communication, wire-like communication, near-field communication (such as inductive coupling between coils of wire or capacitive coupling between conductive electrodes), and far-field communication (such as transferring energy via electromagnetic radiation (e.g., radio waves)). Example communication protocols can include Wi-Fi, Bluetooth®, ZigBee®, Z-wave®, cellular telephony, such as long-term evolution (LTE) and/or 1G, 2G, 3G, 4G, 5G, etc., infrared, radio frequency identification (RFID), satellite transmission, inductive coupling, capacitive coupling, proprietary protocols, combinations of the same, and the like.

The computing device 403 may comprise one or more of a tablet, a PDA, a computer, a laptop, a smartphone, a wearable device, a smartwatch, a database, a sensor, or the like. The computing device 403 can include one or more hardware computer processors.

The one or more audio devices 401 can comprise an auricular device such as any of the example auricular devices shown and/or described herein. The one or more audio devices 401 can emit sound based on audio signals. The one or more audio devices 401 can include one or more speakers. The one or more audio devices 401 can include one or more hardware computer processors. The one or more audio devices 401 can comprise auricular devices, headphones, earphones, earbuds, hearing aids, speakers, sound systems, sound bars, stereo systems, mobile audio devices, portable audio devices, stationary audio devices, mounted audio devices, vehicular audio devices, and the like. In some implementations, one or more of the one or more audio devices 401 can be located in a same location, such as in a same room, or same vehicle. In some implementations, one or more of the one or more audio devices 401 can be remote to each other. In some implementations, one or more of the one or more audio devices 401 can be a single device or system. For example, one audio device 401 may be speaker associated with a left ear of a user and another audio device 401 may be associated with a right ear of a user.

The server 405 can comprise one or more computing devices including one or more hardware processors. The server 405 can comprise program instructions configured to cause the server 405 to perform one or more operations when executed by the hardware processors. The server 405 can include, and/or have access to (e.g., be in communication with and/or host) a storage device, database, or system which can include any computer readable storage medium and/or device (or collection of data storage mediums and/or devices), including, but not limited to, one or more memory devices that store data, including without limitation, dynamic and/or static random-access memory (RAM), programmable read-only memory (PROM), erasable programmable read-only memory (EPROM), electrically erasable programmable read-only memory (EEPROM), optical disks (e.g., CD-ROM, DVD-ROM, etc.), magnetic disks (e.g., hard disks, floppy disks, etc.), memory circuits (e.g., solid state drives, random-access memory (RAM), etc.), and/or the like. In some implementations, the server 405 can include and/or be in communication with a hosted storage environment that includes a collection of physical data storage devices that may be remotely accessible and may be rapidly provisioned as needed (commonly referred to as “cloud” storage). Data stored in and/or accessible by the server 405 can include physiological data, audiometry data, hearing transfer functions, user data, user identification data, device identification data, user preferences, medical data, medical records, audio playback data, or the like. In some implementations, the server 405 can comprise and/or be in communication with an electronic medical records (EMR). An EMR can comprise a propriety EMR. An EMR can comprise an EMR associated with a hospital. An EMR can store data including medical records.

Any of the audio devices 401 can transmit data to any of the other audio devices 401. Any of the audio devices 401 can transmit data to the computing device 403. Any of the audio devices 401 can transmit data to the server 405. The computing device 403 can transmit data to any of the audio devices 401. The computing device 403 can transmit data to the server 405. The server 405 can transmit data to the computing device 403. The server 405 can transmit data to any of the audio devices 401. Data communicated between the audio devices 401, computing device 403, and/or server 405 can include physiological data. Data communicated between the audio devices 401, computing device 403, and/or server 405 can include user data. Data communicated between the audio devices 401, computing device 403, and/or server 405 can include user identification data, device identification data, user preferences, medical data, medical records, audiometry data, hearing transfer functions, audio playback data, or the like.

System 400 can facilitate easy and rapid communication of hearing transfer functions throughout the system (e.g., between devices). Communicating hearing transfer functions between various devices of system 400 can facilitate a user experience. For example, regardless of which device a user chooses to play audio, the device can employ the hearing transfer function specific to the user to optimize the acoustic listening experience of the user. System 400 may facilitate maintaining an up-to-date hearing transfer function of a user across a plurality of devices. If a particular audio device 401A is used to measure updated physiological data (e.g., updated OAE information) or to determine an updated hearing transfer function for a user, that information can be sent to other audio devices 401B and 401C so that they can use the updated information, and the updated information can be stored in the server 405, in some cases. In some cases, the updated information can be sent to the computing device 403, which can use the information to determine an updated hearing transfer function (e.g., from updated OAE information).

In some implementations, an audio device 401 can communicate data, such as hearing transfer function directly to another audio device 401. For example, audio device 401A may comprise a wearable audio device, such as an earbud and audio device 401B may comprise one or more audio devices associated with an audio system of a vehicle. The earbud audio device 401A may communicate data such as one or more hearing transfer functions to the vehicle audio device 401B. Advantageously, a user may experience an improved auditory experience tailored to the user's specific auditory profile whether using the wearable audio device 401A or the vehicle audio device 401B. In some implementations, the wearable audio device 401A may communicate a hearing transfer function to the vehicle audio device 401B in response to a manual user input via the wearable audio device 401A and/or the vehicle audio device 401B. For example, a user may press a button on the wearable audio device 401A and/or the vehicle audio device 401B to cause the wearable audio device 401A to communicate the hearing transfer function to the vehicle audio device 401B. As another example, the wearable audio device 401A may communicate the hearing transfer function to the vehicle audio device 401B in response to mechanically and/or electrically coupling the wearable audio device 401A with the vehicle audio device 401B. For example, a vehicle may comprise a connector port, which can comprise a device cradle or holder or receptacle, or other receiver configured to mechanically receive the wearable audio device 401A (or a housing of the wearable audio device 401A, such as an earbud case). The wearable audio device 401A may automatically communicate a hearing transfer function to the vehicle audio device 401B in response to being mechanically and/or electrically coupled to the connector port of the vehicle. As an example illustration, a user may place their earbuds (or earbud housing or case) into a receptacle of a vehicle which can mechanically receive the earbuds. This may cause the earbud to electrically couple to a computing device of the vehicle and to transmit one or more hearing transfers functions to the vehicle audio system so that the user can enjoy an improved audio experience when listening to audio in their vehicle audio system. Other examples implementation can include communicating hearing transfer functions from other example audio devices such as wearable devices or mobile devices, such as phones.

In some implementations, an audio device 401 can communicate data, such as hearing transfer function, (e.g., directly) to another audio device 401 based on location. For example, an audio device 401 may communicate data in response to determining that another audio device 401 is within a threshold proximity. For example, audio device 401A may communicate one or more hearing transfer functions to audio device 401B when audio device 401B is within a threshold proximity to audio device 401A. An audio device 401 may determine a proximity of another audio device 401 based on a signal strength. In some embodiments, the audio device 401A can determine a quantified distance between the audio device 401A and the other audio device 401B to determine the proximity. In some cases, the audio device 401A can determine the proximity of the other audio device 401B by determining that the other audio device 401B is sufficiently close for communication of information between the audio devices 401A and 401B. In some cases, the audio device 401A can determine the proximity of the 401B device by determining that a wired connection has been established between the audio devices 401A and 401B. In some implementations, audio devices 401 may communicate with each other via one or more wireless communication protocols such as Bluetooth, near field communication (NFC), WiFi, etc. In some implementations, an audio device 401 may communicate data to another audio device based on a proximity and based on determining that the audio devices 401 are associated with each other, such as both being associated with a same user. In some implementations, an audio device 401 may communicate data to another audio device in response to establishing a Bluetooth pairing and/or a Bluetooth connection between the devices.

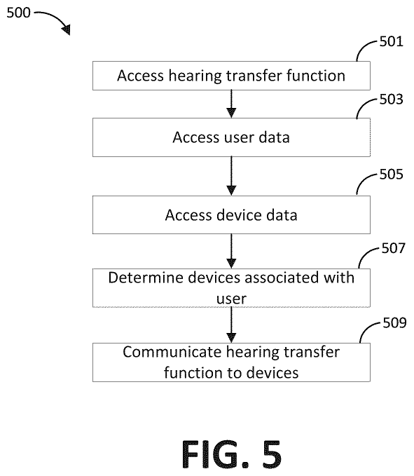

FIG. 5 is a flowchart illustrating an example process 500 of communicating hearing transfer function between audio devices. This process, in full or parts, can be executed by one or more hardware processors, whether they are associated with a singular or multiple computing devices, and even devices in remote or wireless communication. By way of example, the one or more hardware processors executing process 500 can be associated with auricular device 100/200, audio device 401, computing device 403, and/or server 405. The implementations of this process may vary and can involve modifications like omitting blocks, adding blocks, and/or rearranging the order of execution of the blocks. Process 500 serves as an example and is not intended to restrict the present disclosure.

At block 501, a computing device (e.g., one or more hardware processors of a computing device executing program instructions) can access a hearing transfer function associated with a user. In some implementations, the computing device can access more than one hearing transfer function such as one hearing transfer function per ear of a user. The computing device can retrieve the hearing transfer function from memory. The computing device can receive the hearing transfer function from another device such as an audio device. The computing device can access one or more medical data or medical records to retrieve and/or determine the hearing transfer function. The computing device can determine the hearing transfer such as by performing one or more audiometry tests or by accessing audiometry data from a previously performed audiometry test. In some implementations, accessing the hearing transfer function may comprise determining the hearing transfer function based on data, such as auditory profile information, physiological data, or audiometry data, received from another computing device, such as a remote audio device. In some implementations, the hearing transfer function may be an updated hearing transfer function. In some implementations, the hearing transfer function may be a transfer function of the user that was determined most recently compared to other hearing transfer functions of the user.

At block 503, the computing device can access user data. The user data can include account information associated with a user. The user data can include user identification data. The user data may be managed by the user. The user data may be specific to the user. The user data may enable identification of the user. The user data can include an email account, a username, a password, physiological data, an identification number, or the like.

At block 505, the computing device can access device data. The device data can be associated with computing devices. The device data can be associated with audio devices. The device data can enable identification of a device. The device data can include device addresses. The device data can include IP addresses. The device data can include MAC addresses. The device data can include pairing data. The device data can include Bluetooth pairing data.

At block 507, the computing device can determine one or more devices associated with the user. The computing device can determine whether devices are associated with the user based on at least the user data and the device data. For example, the computing device may have access to information relating to whether device data has been associated with user data. As an example, the computing device may access a look up table or other data which may indicate whether device data is associated with user data or vice versa. Devices which the computing device may determine as associated with a user can include audio devices 401 shown and/or described herein. Devices which the computing device may determine as associated with a user can include computing device 403 shown and/or described herein.

At block 509, the computing device can communicate the hearing transfer function accessed at block 501 to the devices determined to be associated with the user. In some implementations, the computing device can communicate more than one hearing transfer function such as one hearing transfer function per ear of a user. Devices to which the computing device may communicate the transfer function can include audio devices 401 shown and/or described herein. Devices to which the computing device may communicate the transfer function can include computing device 403 shown and/or described herein.

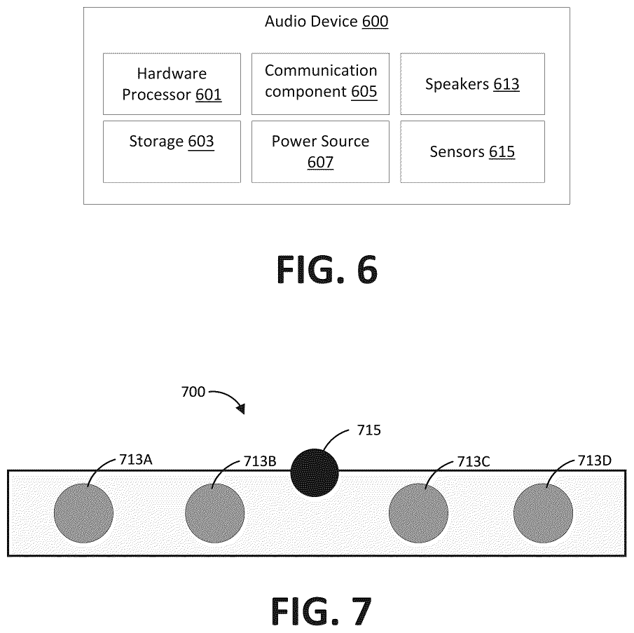

FIG. 6 is a block diagram illustrating an example audio device 600. The audio device can include one or more hardware processors 601, storage 603, a communication component 605, a power source 607, speakers 613, and one or more sensors 615.

The audio device 600 may be a soundbar or other speaker system. The audio device 600 may be a single integrated unit. For example, the various components of audio device 600 may be disposed within a same housing. The audio device 600 may comprise a system of various components remote to one another. For example, one or more of the components shown in audio device 600 may be remote to one or more other components of audio device 600. In some implementations, the audio device 600 may comprise less than all of the components illustrated in FIG. 6. In some implementations, the audio device 600 may comprise additional components than what is illustrated in FIG. 6. The audio device 600 may include similar structural and/or operational features as auricular device 200 shown and/or described herein.

The hardware processor 601 may include similar operational and/or structural features as hardware processor 201 shown and/or described herein. The storage 603 may include similar operational and/or structural features as storage 203 shown and/or described herein. The communication component 605 may include similar operational and/or structural features as communication component 205 shown and/or described herein. The power source 607 may include similar operational and/or structural features as power source 207 shown and/or described herein. The speakers 613 may include similar operational and/or structural features as speakers 213 shown and/or described herein. The sensors 615 may include similar operational and/or structural features as sensors 215 shown and/or described herein.

The speakers 613 can include a plurality of speakers. The speakers 613 can include a speaker array. The speakers 613 may be integrated within a same housing. The speakers 613 may be integrated within a soundbar. One or more of the speakers 613 may be remote to each other. One or more of the speakers 613 may be disposed within separate housings or separate devices.

The sensors 615 can include one or more cameras. The sensors 615 can include an infrared camera. The sensors 615 can include a 3D camera. The sensors 615 can include a stereo camera. The sensors 615 can include an emitter and detector. The sensors 615 can include one or more transceivers. The sensors 615 can include a radio frequency-based sensor. The sensors 615 can include a microwave frequency-based sensor. The sensors 615 can include a radar sensor. The sensors 615 can be configured to emit and/or detect electromagnetic radiation between about 3 GHz to about 300 GHz. The sensors 615 can include a millimeter wave (mmWave) sensor. The sensors 615 can include an ultra-wideband (UWB) sensor. The sensors 615 can include a Lidar sensor. The sensors 615 can include one or more image sensors. The sensors 615 can include an optical sensor. The sensors 615 can include a motion sensor. The sensors 615 can generate image data. The sensors 615 can generate image data responsive to capturing one or more images. The sensors 615 can generate video data. The sensors 615 can generate thermal data. The sensors 615 can generate infrared data. The sensors 615 can generate spatial, directional, and/or depth data. As used herein, sensor data may refer to data generated by the sensors 615.

The communication component 605 can implement one or more wireless communication protocols such as Bluetooth and/or WiFi. The communication component 605 can transmit or receive data such as sensor data.

The hardware processor 601 can access data generated by the sensors 615. The hardware processor 601 can process data generated by the sensors 615. The hardware processor 601 can implement one or more image processing techniques to process and/or analyze the sensor data. The hardware processor 601 can perform pattern recognition on image data generated by the sensor 615. The hardware processor 601 can analyze image data generated by the sensors 615 to identify one or more of size, shape, color, patterns, texture, and/or positions of one or more pixels within images, such as to identify objects and/or persons. The hardware processor 601 can perform facial recognition on image data generated by the sensor 615. The hardware processor 601 can perform gait recognition on image data generated by the sensor 615. The hardware processor 601 can analyze sensor data to determine a thermal signature. The hardware processor 601 can analyze sensor data to determine eyeball detection and/or tracking. The hardware processor 601 can analyze sensor data to identify one or more users. The hardware processor 601 can analyze sensor data to determine the identity of one or more users. The hardware processor 601 can analyze sensor data to identify the presence of one or more users in a given area. The hardware processor 601 can analyze sensor data to determine the location of one or more users in a given area.

The hardware processor 601 can access data from the communication component 605. The hardware processor 601 can process data from the communication component 605. The hardware processor 601 can analyze data from the communication component 605 to determine the location of one or more communication devices, such as phones, tablets, smart devices, wearable devices, auricular devices, etc. The hardware processor 601 can infer the location of one or more users based on the determined location of one or more communication devices. A user may carry a communication device in their hand. A user may wear a communication device. The communication device can be a phone (e.g., a smartphone), a watch (e.g., a smartwatch), a tablet, a wearable device, or the like.

The hardware processor 601 can perform one or more signal processing techniques. The hardware processor 601 can perform beamforming signal processing. The hardware processor 601 can generate beamforming data. Beamforming data can include data relating to amplitude of an audio signal or associated sound. Beamforming data can include data relating to a phase of an audio signal or associated sound. Beamforming data can include data relating to timing or delay of an audio signal or associated sound. Beamforming data can include data relating to a desired direction of an audio signal or associated sound. The beamforming data can include a target location for the sound (e.g., a user's listening location). The beamforming data can include parameters (e.g., filters, signal timing or delay, signal attenuation) that can be applied to modify one or more audio signals presented to one or more speakers to produce audio that is different at a first special location than at a second special location, such as according to an interference pattern, beam pattern, or audio lobes, for example.

The hardware processor 601 can control operation of the speakers 613. The hardware processor 601 can control the phase of audio signals used to output sound from the speakers 613. The hardware processor 601 can control amplitudes of audio signals used to output sound from the speakers 613. The hardware processor 601 can control time delay or other timing of audio signals output from the speakers 613. The hardware processor 601 can control the audio signals used to output sound from the speakers 613 to generate an acoustic beam pattern. The hardware processor 601 can control the audio signals used to output sound from the speakers 613 to control direction of the sound. The hardware processor 601 can control an amplitude and/or phase or timing of audio signals output from speakers 613 to generate a phased array or a steered array. The hardware processor 601 can control an amplitude and/or phase or timing of audio signals output from speakers 613 to generate a desired interference pattern (e.g., constructive and/or destructive interference) between individual audio signals of the various speakers 613. The system can produce interference patterns that produce sound at a target location that is tailored to an auditory profile (e.g., a user-specific auditory profile). The system can produce interference patterns that produce sound at multiple target locations with multiple different associated auditory profiles (e.g., specific to multiple users).

The hardware processor 601 can access one or more transfer functions. The hardware processor 601 can adjust audio playback data to output sound from the speakers 613 according to a transfer function. The hardware processor 601 can adjust audio playback data to output sound from the speakers 613 according to a transfer function and also according to beamforming data. For example, the hardware processor 601 can adjust audio playback data according to a transfer function by applying frequency dependent gains to audio playback data and can further adjust a phase and/or further adjust the amplitude of the audio playback data according to beamforming signal processing techniques to generate a steered array using audio signals to output sound from the speakers 613. The audio playback data that is adjusted according to the transfer function (e.g., tailored to a user's auditory profile) can be modified and sent to multiple speakers 613 to implement beamforming to deliver the adjusted sound to a specific location or area.

FIG. 7 illustrates an example audio device 700. Audio device 700 can include similar structural and/or operational features as audio device 600 shown and/or described herein. Audio device 700 may be a soundbar. Audio device 700 can include one or more speakers 713 such as speaker 713A, speaker 713B, speaker 713C, and speaker 713D. Audio device 700 can include fewer than four speakers. Audio device 700 can include more than four speakers. Speakers 713 may form a speaker array. Speakers 713 may output sound based on one or more audio signals. Audio device 700 can include a sensor 715. The sensor 715 can include similar structural and/or operational features as sensor 615 shown and/or described herein. Sensor 715 may be a camera.

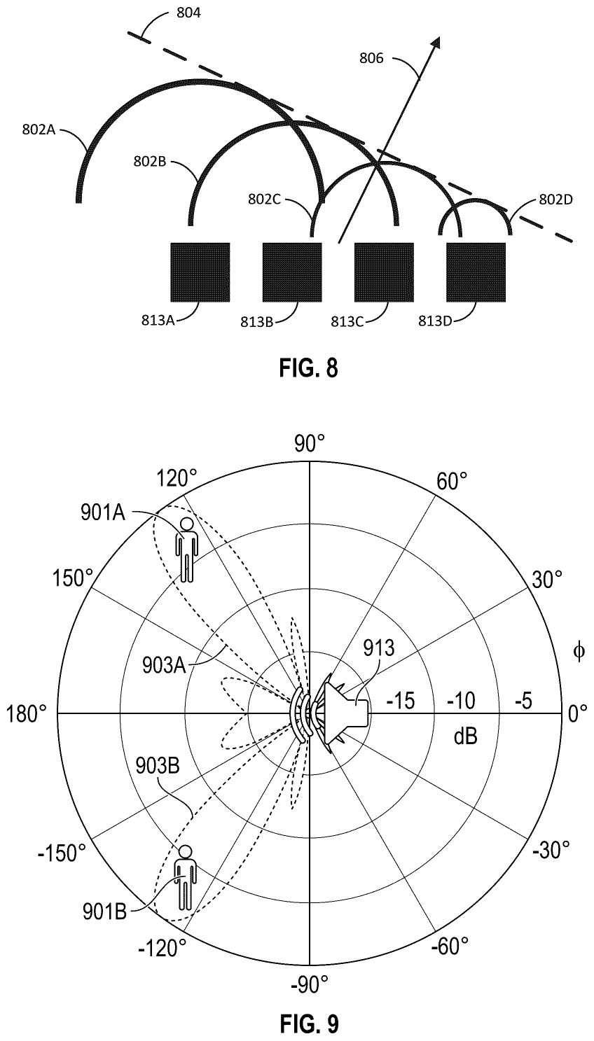

FIG. 8 is a block diagram illustrating an example implementation of speakers 813 (e.g., speakers 813A-813D). Speakers 813 can include similar structural and/or operational features as speakers 613 or speakers 713 shown and/or described herein. In some implementations, speakers 813 may be incorporated into a single device, such as a soundbar. In some implementations, one or more of the speakers 813 may be integrated in separate devices. Speakers 813 can emit sound based on audio playback data. Speaker 813A can emit sound 802A (e.g., based on a first audio playback data). Speaker 813B can emit sound 802B (e.g., based on a second audio playback data). Speaker 813C can emit sound 802C (e.g., based on a third audio playback data). Speaker 813D can emit sound 802D (e.g., based on a fourth audio playback data).

The sounds 802A-802D can interact with each other. Sounds 802A-802D can constructively interfere with each other to increase an amplitude of an acoustic wave (e.g., at a target location or area). Sounds 802A-802D can destructively interfere with each other to decrease an amplitude of an acoustic wave (e.g., at a target location or area).

Speakers 813 can emit sounds of varying amplitude and/or phase. In some cases, multiple speakers 813 may emit sounds having a same frequency. As an example, speaker 813A can emit sound 802A before speaker 813B emits sound 802B, which in turn is before speaker 813C emits sound 802C, which in turn is before speaker 813D emits sound 802D. Sounds 802A-802D that have a same frequency. Sounds 802A-802D may constructively interfere to form a combined audio wave which may be represented by wave plane 804, for example. Speakers 813 can emit the sounds 802 to generate the wave plane 804 in a direction 806. The speakers 813 can adjust one or more of phase and/or amplitude of the sounds 802 to change a direction 806 of the wave plane 804. The speakers 813 can change the direction 806 of the wave plane 804 without mechanically moving speakers 813.

The audio 802A-802D may interact with each other to form an interference pattern (e.g., produced by a steered array or phased array). Wave plane 804 may be referred to as a beam pattern or targeted beam. Wave plane 804 may be referred to as an acoustic lobe. A majority of the acoustic energy may be in the direction 806 of the wave plane 804. The system can change the direction the acoustic energy of wave plane 804 such as be adjusting audio playback data to adjust phase and/or amplitude of the sounds 802A-802D. Various types of interference patterns can be produced. In some cases, a plane wave 804 can be produced as shown in FIG. 8, although in other examples no plane wave 804 is generated.

FIG. 9 is a schematic diagram illustrating an audio device 913 emitting an example beam pattern having acoustic lobes 903A, 903B. Acoustic lobes 903A, 903B may be produced by a steered array, for example. Acoustic lobes 903A, 903B may be the main lobes and may have a higher energy than other lobes. Audio emitted from audio device 913 may have a maximum energy within acoustic lobe 903A and/or acoustic lobe 903B. In some implementations, audio device 913 may emit one acoustic lobe. In some implementations, audio device 913 may emit more than two acoustic lobes. The audio device 913 can emit acoustic lobes 903 at an angle with respect to each other and/or with respect to the audio device 913. Acoustic lobe 903A may be produced by different audio than acoustic lobe 903B. The audio that produces acoustic lobe 903A may include different frequencies than the audio that produces acoustic lobe 903B. Acoustic lobe 903A may be produced using similar audio as those that produce acoustic lobe 903B, in some cases. The audio that produces acoustic lobe 903A may include the same frequencies as the audio that produces acoustic lobe 903B, in some implementations.

As shown, user 901A may be located within acoustic lobe 903A. Acoustic lobe 903A may envelope user 901A. User 901A may hear the sound emitted from audio device 913 within acoustic lobe 903A. User 901A may not hear or may have difficulty hearing the sound emitted from audio device 913 within acoustic lobe 903B. This may be because the audio of acoustic lobe 903B may have minimal energy or imperceptible energy to humans or attenuated energy outside of acoustic lobe 903B.

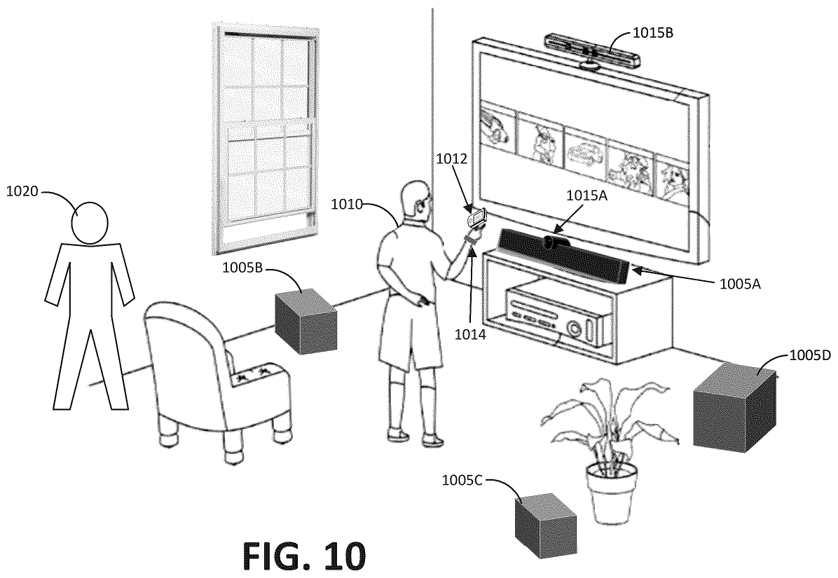

FIG. 10 illustrates an example implementation of an acoustic system including one or more audio devices 1005 (e.g., audio devices 1005A, 1005B, 1005C, 1005D). The audio devices 1005 can include similar structural and/or operational features as any of the other example audio devices shown and/or described herein such as audio device 600 or audio device 700. The audio devices 1005 can comprise a soundbar. The audio devices 1005 can comprise one or more speakers. Audio device 1005A may be a soundbar. Audio device 1005A may include one or more speakers. Audio devices 1005 may be in communication with each other. Audio devices 1005 may communicate wirelessly.

Audio device 1005A can include one or more sensors 1015A. Sensor 1015A can include similar structural and/or operational features as sensor 615 or sensor 715 shown and/or described herein. In some implementations, a sensor 1015B may be remote to the audio device 1005A. The sensor 1015B may communicate with the audio device 1005A, such as wirelessly. Sensor 1015B can include similar structural and/or operational features as sensor 1015A.

Sensors 1015 can collect data relating to the environment. For example, sensors 1015 can collect, image data, video data, infrared data, electromagnetic radiation, etc. A processor, such as may be disposed in audio device 1005A, or remote to audio device 1005A, may process data collected by the sensors 1015. The processor can process the sensor data to determine a location of a user 1010. The processor can process the sensor data to determine an identity of a user 1010. The processor can continually track the location and/or identify of the user 1010 as the user moves about the environment. The processor can process the sensor data to determine a location of a communication device such as wearable device 1014 and/or phone 1012. The processor can process the sensor data to determine an identity of a communication device such as wearable device 1014 and/or phone 1012. The processor may infer the location of the user 1010 based on at least the location and/or identity of the communication device (e.g., wearable device 1014, phone 1012). The processor can determine the location of the wearable device 1014 and/or phone 1012 based on a wireless signal strength from said devices.

Audio devices 1005 can form one or more beam patterns, such as having acoustic lobes (e.g., using a steered array), for example. Audio devices 1005 may form beam patterns individually. For example, each audio device 1005 may comprise a speaker array configured to generate a sound according to a beam pattern. Audio devices 1005 may operate in conjunction to form one or more beam patterns. For example, each audio device 1005 may comprise a speaker of a speaker array formed by the combination of audio devices 1005. Audio devices 1005 can form one or more steered arrays as shown and/or described in FIGS. 8-9.

Audio devices 1005 can adjust a direction of acoustic lobes and/or can adjust the location of one or more listening areas (e.g., which can received audio based on different auditory profiles). Audio devices 1005 can adjust a direction of acoustic lobes or listening locations without changing physical location or position. The audio devices 1005 may adjust the direction of acoustic lobes or locations of listening locations based at least on the location of the user 1010. A processor can analyze sensor data collected by sensors 1015 to determine a user 1010 location. The processor can cause the audio devices 1005 to adjust one or more of phase or timing and/or amplitude of the audio signals to change a direction of sound output from a steered array. The audio devices 1005 may emit the sound in the direction of the user's 1010 determined location as the user 1010 moves about the environment. The user 1010 may be continually within the acoustic lobe or listening location as the user 1010 moves about the environment. The user 1010 may experience an optimal acoustic experience as the user 1010 moves about the environment at least because the user 1010 is continually within the acoustic lobe or tailored listening area.

Audio devices 1005 can generate acoustic lobes or listening area for different users. Audio devices 1005 can generate an acoustic lobe or listening area for user 1010 and can generate a different acoustic lobe or listening area for user 1020. In some implementations, audio devices 1005 can generate an acoustic lobe or listening area for user 1010 but not for user 1020. User 1020 may not hear, or may have difficulty hearing, audio corresponding to the acoustic lobe or listening area associated with user 1010 at least because user 1020 is outside the acoustic lobe or listening area associated with user 1010. User 1010 may not hear, or may have difficulty hearing, audio corresponding to the acoustic lobe or listening area associated with user 1020 at least because user 1010 is outside the acoustic lobe or listening area associated with user 1020. Audio devices 1005 can simultaneously generate different acoustic lobes or listening areas for different users.

Audio devices 1005 can emit audio signals according to one or more transfer functions. Transfer functions can include hearing transfer functions associated with various users. For example, audio devices 1005 can use audio playback data according to a hearing transfer function associated with user 1010 and can use audio playback data according to a different hearing transfer function associated with user 1020. Audio devices 1005 can adjust audio playback data according to a transfer function to emit audio according to the transfer function. Example implementation of adjusting audio playback data according to transfer functions may be shown and/or described in greater detail with respect to FIG. 3. Audio devices 1005 can simultaneously emit audio according to a plurality of transfer functions. Accordingly, user 1010 may listen to audio optimized for user 1010 according to a transfer function associated with user 1010 simultaneously as user 1020 listens audio optimized for user 1020 according to a transfer function associated with user 1020.

Transfer functions can include environmental transfer functions associated with an environment. A processor, such as a processor disposed within audio device 1005A, can determine one more acoustic characteristics of an environment. The processor can determine the acoustic characteristics of the environment based on at least sensor data. As an example, the processor can determine a spectral response of the environment. The processor can perform an in-room equalization. The processor can determine the environmental transfer function based on at least early arrivals or late arrivals (also referred to as “reverberance”). The audio devices 1005 can adjust emitted audio signals according to an environmental transfer function to account for acoustic characteristics of the environment such as early arrivals or late arrivals. Acoustic characteristics of an environment can be affected by a size of the environment, objects within the environment, such as plants, furniture, people, etc., material of objects within the environment, acoustic reflectivity of surfaces within the environment, whether the environment is open, partially open, or closed, such as whether a window or door is ajar, etc. As an example, the processor may cause the audio devices 1005 to adjust a base gain depending on the size of a room (e.g., suppress low frequency amplitudes in a small room or amplify low frequency amplitudes in a large room).

Audio devices 1005 can implement beam patterns or transfer functions alone or in combination. For example, the audio devices 1005 can emit audio to generate a particular acoustic lobe of a beam pattern and according to one or more hearing transfer functions, such as a hearing transfer function or an environmental transfer function. As another example, the audio devices 1005 can emit audio according to user hearing transfer function and according to an environmental transfer function. Accordingly, the audio devices 1005 can emit audio directed toward a particular location (e.g., a user location) and that is optimized based on the user's particular auditory profile and/or the particular acoustic characteristics of the environment.

FIG. 10 is shown as an example and is not intended to be limiting of the present disclosure. The environment depicted in FIG. 10 could be an acoustic environment, including for example, a room, a vehicle, a movie theater, a stadium, an arena, a concert hall, an indoor environment, an outdoor environment, or the like.

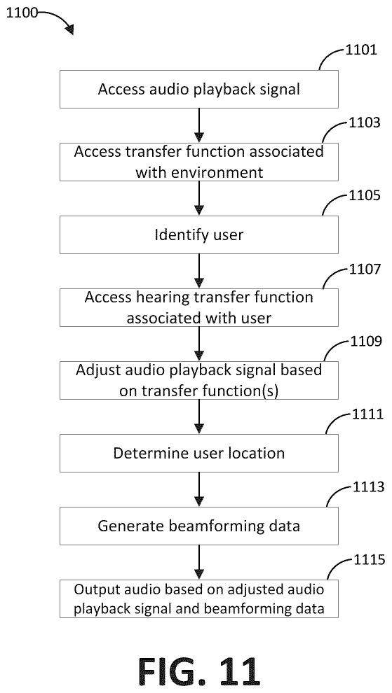

FIG. 11 is a flowchart illustrating an example process 1100 for optimizing audio. Process 1100, or portions thereof, can be performed by one or more hardware computer processors, such as any of the example processors shown and/or described herein. In some implementations, hardware processor 201 shown and/or described herein can perform one or more portions of process 1100. Process 1100 is provided as an example and is not intended to be limiting of the present disclosure. In some implementations, the processor can omit portions of the process 1100, may add additional operations, and/or may rearrange an order in which the operations of the process 1100 are executed.

At block 1101, a computing device (e.g., one or more hardware processors of a computing device executing program instructions) can access an audio playback signal (which may also be referred to as audio playback data). The processor can access the audio playback signal from memory. The computing device can receive the audio playback signal (e.g., wirelessly) via a network. The audio playback signal can correspond to a sound desired to be reproduced. The audio playback signal can include music or media.

At block 1103, the computing device can optionally access an environmental transfer function or filter associated with the environment. Accessing the environmental transfer function or filter can include retrieving the environmental transfer function or filter from memory. Accessing the environmental transfer function or filter can include receiving the environmental transfer function or filter from a remote device, such as wirelessly over a network. Accessing the environmental transfer function or filter can include determining the environmental transfer function or filter. The computing device can determine the environmental transfer function or filter based on at least one or more acoustic characteristics of the environment. The computing device can determine the environmental transfer function based on at least a spectral response of the environment. The computing device can determine the environmental transfer function based on at least performing an “in-room equalization” of the environment. In some implementations, the computing device can determine a plurality of environmental transfer functions. In some implementations, block 1103 can be omitted, such as if the device or system does not adjust the audio signals according to the environment.

At block 1105, the computing device can identify a user. Identifying the user can include determining the presence of user such as whether the user is in an environment or not. Identifying a user can include determining the identity of a user such as to distinguish a plurality of users within a same environment. The computing device can identify the user based on at least sensor data. As an example, the computing device can analyze image data collected by a camera, such as by performing image processing, to identify a user. The user can be identified in any suitable manner, as discussed herein.

At block 1107, the computing device can access a hearing transfer function associated with the identified user. Accessing the hearing transfer function can include retrieving the hearing transfer function from memory. Accessing the hearing transfer function can include receiving the hearing transfer function from a remote device, such as wirelessly over a network. Accessing the hearing transfer function can include determining the hearing transfer function from audiometry data generated from an audiometry test. In some cases, the hearing transfer function can include a filter, which can for example be applied to an audio signal (e.g., the audio playback signal). The computing device can determine the hearing transfer function based on at least physiological data associated with the user such as audiometry data, medical records, or the like. The computing device can receive the physiological data from memory. The computing device can receive the physiological data from a remote computing device such as a server, database, or audio device. In some implementations, the computing device can determine more than one hearing transfer function associated with the user such as one hearing transfer function associated with each ear of the user.

The computing device can perform any of blocks 1105-1107 for multiple users. The computing device can perform any of blocks 1105-1107 for multiple users simultaneously.

At block 1109, the computing device can adjust the audio playback signal based on one or more transfer functions. The transfer function(s) can include one or more hearing transfer functions associated with a user. The transfer function(s) can include an environmental transfer function. The computing device can adjust an amplitude of the audio playback signal. The computing device can adjust a phase or timing of the audio playback signal. The computing device can adjust a latency of the audio playback signal. The computing device can make frequency-specific adjustments to amplitude of the audio playback signal. The computing device can apply one or more frequency dependent gains to the amplitude of the audio playback signal. The computing device can make frequency-specific adjustments to a phase of the audio playback signal. The computing device can apply one or more filters to the audio playback signal. The computing device can adjust the audio playback signal by inserting additional audio signals which may interfere (e.g., constructively or destructively) with the audio playback signal).

At block 1111, the computing device can determine a location of a user within an environment. The computing device can perform block 1111 for multiple users, including simultaneously. The computing device can determine the user location based on at least sensor data (e.g., such as image data and/or radar data). As an example, the computing device can analyze image data collected by a camera to determine a user location within an environment. As another example, the computing device can implement one or more radio frequency-based protocols, such as ultra-wideband (UWB) and/or millimeter wave (mm Wave) to determine the user location. The computing device can use a Lidar system to identify a user and/or to determine a location of the user. Other presence or location sensors can be used. In some implementations, the locations of users may be predetermined. For example, the locations of seats (e.g., in a vehicle, in a theater, etc.) may be fixed relative to the locations of audio speakers. The computing device can access the predetermined, fixed user locations from storage such that the computing device may not need to determine the location from sensor data which can reduce processing times and improve computational efficiency. As an example, the computing device can determine the identity of a user that is in a predetermined location of a driver's seat in a vehicle and can determine the identity of another user in another predetermined location in a passenger seat of the vehicle. The computing device can generate audio for the user in the driver's seat and audio for the other user in the passenger seat as described in subsequent blocks without having to determine specific locations because the driver's seat location and the passenger seat location are fixed and predetermined.

At block 1113, the computing device can generate beamforming data. Beamforming data can include data relating to generating a beam pattern. Beamforming data can include data relating to modifying the audio playback signal, including a phase and/or amplitude of the audio playback signal. The computing device can generate the beamforming data based on at least the user location. The beamforming data can generate audio having one or more acoustic lobes that correspond to the user locations. In some implementations, the computing device can continually update the beamforming data based on the user location as the user moves throughout the environment such that the location of the acoustic lobe continually tracks the location of the user. In some implementations, the beamforming data can generate audio having acoustic lobes at fixed locations such as in situations where the user locations are predetermined/fixed as described above (e.g., seats in a vehicle). In such cases, the computing device may not need to update the beamforming data to change the location of the acoustic lobes because the user locations may not change. For example, the computing device can access predetermined beamforming data that corresponds to predetermined user locations. Accessing predetermined beamforming data without having to continually compute new beamforming data may reduce processing requirements and improve processing speeds. The computing device can generate beamforming data for one or more speakers. For example, the computing device may generate beamforming data for each speaker in a speaker array. The computing device can generate different beamforming data for different speakers.

At block 1115, the computing device can cause one or more speakers to output audio based on the modified audio playback signal and the beamforming data. The one or more speakers can output sound based on the modified audio playback signal according to a beam pattern dictated by the beamforming data. The one or more speakers can output sound based on the modified audio playback signal using a steered array, for example as dictated by the beamforming data. The one or more speakers can provide audio to the user location, and that audio can be different from other locations in the listening space. For example, the audio delivered to the user location can be modified audio based at least in part on the user auditory profile information (e.g., the one or more hearing transfer functions associated with the user). The one or more speakers can provide multiple modified audio experiences at multiple locations, such as when multiple users are in the listening space. For example, as shown in FIG. 9, a first modified audio (e.g., based on a first user auditory profile) can be provided to the location of a first user 901A (e.g., using the acoustic lobe 903A), and a second modified audio (e.g., based on a second user auditory profile) can be provided to the location of a second user 901B (e.g., using the acoustic lobe 903B). The same one or more speakers (e.g., which can be a steered array) can be used to provide the first modified audio and the second modified audio. For example, first audio signals, which can be configured to provide the first modified audio to the first user location, can be superimposed with second audio signals, which can be configured to provide the second modified audio to the second user location. Both the first modified audio and the second modified audio can be different from the audio provided to other locations in the listening space. Modified audio be sent to additional users in the listening space as well, in like manner.

The computing device can perform any of blocks 1101-1115 iteratively. The computing device can perform iteratively perform blocks 1111-1115. The computing device may continuously determine an updated user location so that the generated beamforming data is accurate with the most up-to-date user location.

Although various implementations are disclosed herein as using a hearing transfer function for a user, in some cases, a user auditory profile can be used. In some cases, the user auditory profile can include a hearing transfer function, which can correlate one or more input amplitudes with one or more corresponding perceived amplitude for one or more audio frequencies. In some cases, other types of auditory profile information can be used. For example, an auditory profile for a user can indicate that one or more frequencies or frequency ranges should be amplified or attenuated, such as by a specified degree. This auditory profile information can be conveyed using a transfer function, or any other suitable type of data structure. Accordingly, each example and features that discusses a hearing transfer function could use any other suitable type of auditory profile information.

### Additional Considerations

Certain categories of persons, such as caregivers, clinicians, doctors, nurses, and friends and family of a user, may be used interchangeably to describe a person providing care to the user. Furthermore, patients or users used herein interchangeably refer to a person who is wearing a sensor or is connected to a sensor or whose measurements are used to determine a physiological parameter or a condition. Parameters may be, be associated with, and/or be represented by, measured values, display icons, alphanumeric characters, graphs, gages, power bars, trends, or combinations. Real time data may correspond to active monitoring of a user, however, such real time data may not be synchronous to an actual physiological state at a particular moment. Measurement value(s) of a parameter and the parameter used herein such as, SpO2, RR, PaO2 and the like, unless specifically stated otherwise, or otherwise understood with the context as used is generally intended to convey a measurement or determination that is responsive to the physiological parameter.

Although certain implementations and examples have been described herein, it will be understood by those skilled in the art that many aspects of the systems and devices shown and described in the present disclosure may be differently combined and/or modified to form still further implementations or acceptable examples. All such modifications and variations are intended to be included herein within the scope of this disclosure. A wide variety of designs and approaches are possible. No feature, structure, or step disclosed herein is essential or indispensable. The various features and processes described herein may be used independently of one another, or may be combined in various ways. For example, elements may be added to, removed from, or rearranged compared to the disclosed example implementations. All possible combinations and sub-combinations are intended to fall within the scope of this disclosure.

Any methods and processes described herein are not limited to any particular sequence, and the blocks or states relating thereto can be performed in other sequences that are appropriate. For example, described blocks or states may be performed in an order other than that specifically disclosed, or multiple blocks or states may be combined in a single block or state, or certain method or process blocks may be omitted, or certain blocks or states may be performed in a reverse order from what is shown and/or described. The example blocks or states may be performed in serial, in parallel, or in some other manner. Blocks or states may be added to or removed from the disclosed example implementations.

The methods disclosed herein may include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, and/or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. The computer system may be a cloud-based computing system whose processing resources are shared by multiple distinct entities or other users. The systems and modules may also be transmitted as generated data signals (for example, as part of a carrier wave or other analog or digital propagated signal) on a variety of computer-readable transmission mediums, including wireless-based and wired/cable-based mediums, and may take a variety of forms (for example, as part of a single or multiplexed analog signal, or as multiple discrete digital packets or frames).

Many other variations than those described herein will be apparent from this disclosure. For example, depending on the implementation, certain acts, events, or functions of any of the algorithms described herein can be performed in a different sequence, can be added, merged, or left out altogether (for example, not all described acts or events are necessary for the practice of the algorithms). Moreover, in certain implementations, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

Various illustrative logical blocks, modules, routines, and algorithm steps that may be described in connection with the disclosure herein can be implemented as electronic hardware (e.g., ASICs or FPGA devices), computer software that runs on computer hardware, or combinations of both. Various illustrative components, blocks, and steps may be described herein generally in terms of their functionality. Whether such functionality is implemented as specialized hardware versus software running on general-purpose hardware depends upon the particular application and design constraints imposed on the overall system. The described functionality can be implemented in varying ways for each particular application, but such implementation decisions should not be interpreted as causing a departure from the scope of the disclosure.

Moreover, various illustrative logical blocks and modules that may be described in connection with the implementations disclosed herein can be implemented or performed by a machine, such as a general purpose processor, a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. A general purpose processor can be a microprocessor, controller, microcontroller, or state machine, combinations of the same, or the like. A processor can include electrical circuitry configured to process computer-executable instructions. A processor can include an FPGA or other programmable devices that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor may also include primarily analog components. For example, some, or all, of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of any method, process, routine, or algorithm described in connection with the disclosure herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain features, elements, and/or steps are optional. Thus, such conditional language is not generally intended to imply that features, elements, and/or steps are in any way required or that one or more embodiments necessarily include logic for deciding, with or without other input or prompting, whether these features, elements, and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 10 degrees, 5 degrees, 3 degrees, or 1 degree. As another example, in certain embodiments, the terms “generally perpendicular” and “substantially perpendicular” refer to a value, amount, or characteristic that departs from exactly perpendicular by less than or equal to 10 degrees, 5 degrees, 3 degrees, or 1 degree.

As used herein, “real-time” or “substantial real-time” may refer to events (e.g., receiving, processing, transmitting, displaying etc.) that occur at a same time as each other, during a same time as each other, or overlap in time with each other. “Real-time” may refer to events that occur at distinct or non-overlapping times the difference between which is imperceptible and/or inconsequential to humans such as delays arising from electrical conduction or transmission. A human may perceive real-time events as occurring simultaneously, regardless of whether the real-time events occur at an exact same time. As a non-limiting example, “real-time” may refer to events that occur within a time frame of each other that is on the order of milliseconds, seconds, tens of seconds, or minutes. For example, “real-time” may refer to events that occur within a time frame of less than 1 minute, less than 30 seconds, less than 10 seconds, less than 1 second, less than 0.05 seconds, less than 0.01 seconds, less than 0.005 seconds, less than 0.001 seconds, etc.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

As used herein, “system,” “instrument,” “apparatus,” and “device” generally encompass both the hardware (for example, mechanical and electronic) and, in some implementations, associated software (for example, specialized computer programs for operational control) components.

It should be emphasized that many variations and modifications may be made to the herein-described implementations, the elements of which are to be understood as being among other acceptable examples. All such modifications and variations are intended to be included herein within the scope of this disclosure. Any section headings used herein are merely provided to enhance readability and are not intended to limit the scope of the implementations disclosed in a particular section to the features or elements disclosed in that section. The foregoing description details certain implementations. It will be appreciated, however, that no matter how detailed the foregoing appears in text, the systems and methods can be practiced in many ways. As is also stated herein, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Those of skill in the art would understand that information, messages, and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

What is claimed is:

1. An audio system comprising:
one or more hardware processors configured to:
access a hearing transfer function originating from an auricular device, wherein the hearing transfer function is generated from audiometry data associated with a user, wherein the hearing transfer function is configured to correct audio playback to account for differences between a hearing perception of the user and normal hearing perception;
determine a spatial location of a user within an environment from sensor data;
based on the spatial location of the user, generate beamforming data for generating audio according to a beam pattern having an acoustic lobe at the spatial location of the user;
modify an audio playback signal based on the hearing transfer function; and
cause one or more speakers to emit modified audio based on the modified audio playback signal with the beamforming data to cause the acoustic lobe to form at the spatial location of the user with the modified audio to account for the hearing perception of the user.

2. The audio system of claim 1 wherein the one or more hardware processors are configured to:
determine an updated spatial location of the user within the environment from the sensor data as the user moves throughout the environment; and
generate the beamforming data for generating the audio having the acoustic lobe at the updated spatial location of the user as the user moves throughout the environment.

3. The audio system of claim 1 wherein the one or more hardware processors are configured to:
generate the beamforming data based on the spatial location of the user and another spatial location of another user, the beam pattern having the acoustic lobe at the spatial location of the user and another acoustic lobe at the another spatial location of the another user; and
cause the one or more speakers to emit the audio based on the beamforming data to cause the acoustic lobe to form at the spatial location of the user with the modified audio and to cause the another acoustic lobe to form at the another spatial location of the another user without the modified audio.

4. The audio system of claim 1 wherein the audiometry data includes one or more of DPgram data or audiogram data.

5. The audio system of claim 1 wherein the sensor data originates from one or more of a camera, mmWave sensor, or ultra-wide band (UWB) sensor.

6. The audio system of claim 1 wherein the sensor data comprises image data, wherein the one or more hardware processors are configured to:
determine an identity of the user from the image data with one or more image processing techniques; and
update the beamforming data as the user moves throughout the environment to update the beam pattern with the acoustic lobe continuously at the spatial location of the user.

7. The audio system of claim 1 wherein the one or more hardware processors are configured to:
analyze the sensor data to determine an identity of the user; and
access the hearing transfer function from memory based on determining that the identity of the user corresponds to the hearing transfer function.

8. The audio system of claim 1 wherein the one or more hardware processors are configured to:
access the hearing transfer function from the auricular device over a wireless communication network based on determining that the auricular device and the audio system are associated with the user.

9. The audio system of claim 1 wherein the one or more speakers are positioned in a vehicle, wherein the one or more hardware processors are configured to access the hearing transfer function from the auricular device in response to the auricular device being positioned within a receptacle of the vehicle.

10. The audio system of claim 1 wherein the one or more hardware processors are configured to access the hearing transfer function from an electronics medical records (EMR) database over a network.

11. The audio system of claim 1 wherein the one or more hardware processors are configured to access a hearing transfer function associated with each ear of the user.

12. The audio system of claim 1 wherein the one or more hardware processors are configured to:
access an environmental transfer function associated with an environment of the user, wherein the environmental transfer function is based on one or more acoustic characteristics of the environment determined from an audio spectral response; and
modify the audio playback signal based on the environmental transfer function.

13. The audio system of claim 1 wherein the one or more hardware processors are configured to apply one or more device filters to the audio playback signal to modify the audio playback signal to account for physical characteristics of the audio system that affect an acoustic quality of audio playback from the one or more speakers.

14. The audio system of claim 1 wherein the one or more hardware processors are configured to modify the audio playback signal based on applying one or more frequency dependent gains to an amplitude of the audio playback signal.

15. The audio system of claim 1 wherein the one or more hardware processors are configured to modify the audio playback signal based on adjusting one or more of a phase of the audio playback signal, a latency of the audio playback signal, or an amplitude of the audio playback signal.

16. A computer-implemented method comprising:
accessing a hearing transfer function originating from an auricular device, wherein the hearing transfer function is generated from audiometry data associated with a user, wherein the hearing transfer function is configured to correct audio playback to account for differences between a hearing perception of the user and normal hearing perception;
determining a spatial location of a user within an environment from sensor data;
based on the spatial location of the user, generating beamforming data for generating audio according to a beam pattern having an acoustic lobe at the spatial location of the user;
modifying an audio playback signal based on the hearing transfer function; and
causing one or more speakers to emit modified audio based on the modified audio playback signal with the beamforming data to cause the acoustic lobe to form at the spatial location of the user with the modified audio to account for the hearing perception of the user.

17. The computer-implemented method of claim 16 further comprising:
determining an updated spatial location of the user within the environment from the sensor data as the user moves throughout the environment; and
generating the beamforming data for generating the audio having the acoustic lobe at the updated spatial location of the user as the user moves throughout the environment.

18. The computer-implemented method of claim 16 further comprising:
generating the beamforming data based on the spatial location of the user and another spatial location of another user, the beam pattern having the acoustic lobe at the spatial location of the user and another acoustic lobe at the another spatial location of the another user; and
causing the one or more speakers to emit the audio based on the beamforming data to cause the acoustic lobe to form at the spatial location of the user with the modified audio and to cause the another acoustic lobe to form at the another spatial location of the another user without the modified audio.

19. Non-transitory computer-readable media including computer-executable instructions that, when executed by a computing system, cause the computing system to perform operations comprising:
accessing a hearing transfer function originating from an auricular device, wherein the hearing transfer function is generated from audiometry data associated with a user, wherein the hearing transfer function is configured to correct audio playback to account for differences between a hearing perception of the user and normal hearing perception;
determining a spatial location of a user within an environment from sensor data;
based on the spatial location of the user, generating beamforming data for generating audio according to a beam pattern having an acoustic lobe at the spatial location of the user;
modifying an audio playback signal based on the hearing transfer function; and
causing one or more speakers to emit modified audio based on the modified audio playback signal with the beamforming data to cause the acoustic lobe to form at the spatial location of the user with the modified audio to account for the hearing perception of the user.

20. The non-transitory computer-readable media of claim 19 wherein the computer-executable instructions, when executed by the computing system, cause the computing system to perform operations comprising:
determining an updated spatial location of the user within the environment from the sensor data as the user moves throughout the environment; and
generating the beamforming data for generating the audio having the acoustic lobe at the updated spatial location of the user as the user moves throughout the environment.
